(19) United States
(12) Patent Application Publication
(10) Pub. No.: US 2025/0228604 A1
(43) Pub. Date: Jul. 17, 2025

(54) SYSTEM AND METHOD FOR ELECTRICALLY STIMULATING TISSUE

(71) Applicant: SYNAPSTIM, Herzedia (IL)

(72) Inventors: Sasi SOLOMON, Herzedia (IL); Semion SIMON, Netanya (IL)

(21) Appl. No.: 18/701,599

(22) PCT Filed: Nov. 1, 2022
(86) PCT No.: PCT/IL2022/051156

§ 371 Date: Apr. 15, 2024

Related U.S. Application Data
(60) Provisional application No. 63/273,961, filed on Oct. 31, 2021.

(51) Int. Cl. A61B 18/14; A61B 18/00; A61B 18/12; A61B 90/00; A61N 1/04; A61N 1/36

(52) U.S. Cl. CPC A61B 18/14; A61B 18/1206; A61B 90/06; A61B 2018/00071; A61B 2018/0047; A61B 2018/00577; A61B 2018/00654; A61B 2018/00678; A61B 2018/00696; A61B 2018/00702; A61B 2018/00732; A61B 2018/124; A61B 2018/143; A61B 2090/064; A61N 1/0456; A61N 1/3603

(57) ABSTRACT

A system and method for electrical stimulation of a body tissue. An array of microneedles, formed from an electrically conductive material are adapted for puncturing a surface of the tissue. A processing unit is configured to determine from a signal when the microneedles in the microneedle array are in a predetermined position in the tissue or when tissue surrounding one or more microneedles has been ablated.

### FIELD OF THE INVENTION

The present invention relates to medical devices and more specifically to such devices for electrical stimulation of body tissues.

### BACKGROUND

Electrical stimulation of body tissues is used for treatment of chronic and acute medical conditions and can be carried out using implantable or surface electrodes. Implanted electrodes are typically used to directly stimulate tissue at a desired site. While effective in stimulating target tissues, implanted electrodes exhibit significant drawbacks such as implant site infections, electrode failure and electrode displacement.

Despite improvements to implantable electrode technologies in recent years, transcutaneous electrical stimulation (TES) is still the most frequently applied approach for stimulating muscle and nerve tissue that are close to the skin surface and is commonly used for relieving pain, for rehabilitation and for treating migraine. TES utilizes surface electrodes rather than implanted electrodes for delivering an electrical signal to tissue such as muscle or nerve tissue. Surface electrodes do not require an implantation procedure and hence avoid some of the drawbacks of implanted electrodes. While transcutaneous electrodes overcome some of the above mentioned drawbacks of implantable electrodes, their positioning on the skin surface often diverts the electric signal from the target tissue (e.g., nerves, muscle) and forces the electrical signal to travel through electrically resistive tissue (e.g., keratinized layer of the skin). These drawbacks of surface electrodes often make them unsuitable for stimulation of deep tissue such as the tibial nerve for applications such as overactive bladder or the vagus nerve for applications such as autoimmune and inflammatory diseases.

The largest drawback to using surface electrodes arises from the fact that for many applications, surface electrodes are too far from the tissue to be stimulated to be effective. In the case of surface skin electrodes, the electrical signal will travel through superficial layer of the skin such as the keratinized skin layer where the majority of the signal is dissipated, and the electrical signal will not reach target tissue (nerve or muscle) in the deeper layers.

U.S. Pat. No. 10,688,301 discloses a TES system that employs a microneedle array for forming electrically conductive micropores through a tissue surface. It was found that the formation of micropores in the superficial layers of the skin enhances the penetration of the electrical stimulation into the deeper layers of the tissue. thus overcoming some of the aforementioned drawbacks of standard surface electrode systems. While the system disclosed in U.S. Pat. No. 10,688,301 substantially improves TES, there still remains a need for a system and method for electrically stimulating tissue using surface electrodes.

### SUMMARY OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

In one of its aspects, the present invention provides a system for electrical stimulation of a body tissue. In one of its embodiments, the system of the invention includes an applicator including an array of two or more microneedles. The microneedles are made from an electrically conductive material such as stainless steel, and, in addition to being able to puncture the tissue surface and enter the tissue, the microneedles also function as electrodes.

The system may further include a control unit, which includes a processing unit, a signal generator, power unit and a switching circuit. The processing unit controls the switching circuit, to intermittently connect the various electrodes of the system to poles of the signal generator during the various stages of the treatment

The system may further comprises a force detector that may be incorporated into the applicator. The force detector detects pressure applied to the tissue surface by the tips of the microneedles as the applicator is pressed into the tissue surface during penetration of the microneedles into the tissue.

The system further comprises one or more surface electrodes. The processing unit also controls the switching circuit to intermittently connect two or more surface electrodes to the poles of the power unit and/or the signal generator.

In use, the applicator is applied to a first tissue surface overlying a deeper second tissue region to be treated. The second tissue may be, for example, a nerve or nerve tissue. For example, the applicator can be placed on the neck of an individual over the vagus nerve in a treatment for stimulating the vagus nerve. As pressure is applied by the applicator, the force detector detects a pressure applied to the tissue surface and generates an electrical signal indicative of the pressure that is input to the processing unit. The processing unit constantly monitors or is triggered by the signal input from the force transducer and determines whether the force of the applicator on the tissue surface is at least a threshold pressure. In an alternative embodiment, the force detector closes a switch which sends a signal to the processing unit that a pressure has been detected.

When the pressure of the applicator on the tissue surface is at least a predefined pressure, this is an indication that the microneedles are applying the requisite pressure and this is indicative of the microneedles being in the requisite position. In the case of skin, the microneedles are applying the requisite pressure when the microneedle tips are pressed through the stratum corneum layer of the skin.

Additionally or alternatively some or all of the microneedles may be connected to a pole of the signal generator and a ground electrode applied to the tissue surface. A DC or pulsatile signal of non-stimulating energy (e.g., 1 V or 2 mA) is generated by the signal generator generating an electrical signal between the microneedles and the ground electrode. The current is monitored by the processing unit and the tissue impedance is calculated from the electrical signal. A tissue impedance that is below a predetermined threshold is indicative of the formation of micropores, and that the requisite pressure is being applied.

When it is determined that the requisite pressure is being applied, an ablation signal is generated by the signal generator and applied between a first subset of one or more of the microneedles, on the one hand, and a second subset of microneedles, on the other hand. The ablation signal is designed to cause ablation of the tissue surrounding the microneedles in the first subset. Since tissue is elastic in nature, micropores formed by microneedles tend to partially or fully close when the microneedles are moved. In order to ensure that the micropores formed by the microneedle array remain open following removal of the microneedles the ablative electric signal is used to ensure that the micropores remain open.

As the ablation signal is being applied between the two subsets of microneedles, the processing unit monitors the current flowing between the microneedles of the two subsets and determines if the ablation around the microneedles in the first subset is satisfactory. Satisfactory ablation of the tissue around the microneedles reduces the overall impedance of the tissue and reduces the amplitude of the treatment signal by surface electrodes needed for effective treatment.

The process is repeated one or more times with different selections of the first and second subsets of microneedles. The processing unit then determine whether the overall ablation of the tissue is satisfactory. When it is determined that ablation of the tissue region is satisfactory, the applicator is removed from the tissue surface and a treatment surface electrode is placed on the tissue surface over the micropores that were formed in the tissue. A treatment signal is then generated between the treatment surface electrode passing through a second tissue underlying the tissue surface. The treatment continues as required in any application.

The system may include a communication module for wireless communication to external devices (e.g., smartphone, computer etc.) and/or to a cloud-based database server.

The communication module can be used to store data (also on a cloud-based repository), for software updates, training, remote assistance, and communication between the present system and a physician. The communications module can enable remote access to data and information by a health care professional who could respond and provide instructions in real time.

The system can further include sensors for measuring physiological parameters of the subject such as heart rate, blood pressure, sweat, respiration rate and temperature. These parameters are analyzed to assist in determining the type and/or timing of the electrical signal delivery by the surface electrodes. For example, electrical signal delivery to the vagus nerve is known to influence the blood pressure. Sensing the blood pressure and analyzing it may affect the delivered electrical signal characteristics and timing.

A fluid analysis unit can also be incorporated into/connected to the present system to provide blood, urine, sweat or saliva chemistry. Such an analysis may have an effect on the timing and characteristics of the electrical signal delivery and the location of delivery. For example, cytokines analysis from blood samples may indicate the effectiveness of the treatment and whether there is a need to modify the electrical signal characteristics and/or timing to influence the cytokines level.

The efficacy of electrical conduction from a surface electrode into a tissue through micropores depends on the quality of the micropores (e.g., depth, diameter, shape, structure etc.). The invention allows verification that micropores formed by the microneedle array have the desired characteristics. This in turn helps to maximize patient comfort.

The present system can be used for aesthetic treatment (of, for example, facial skin) or to treat a variety of disorders/conditions that can benefit from electrical stimulation of, for example, muscle or nerve tissue.

The following is an incomplete list of disorders or conditions treatable by the present invention.
(i) Pain relief—back pain, neuropathic pain, carpal tunnel syndrome, shoulder pain, chronic and intractable pain including diabetic neuropathy, complex regional pain syndrome, phantom limb pain, ischemic limb pain, refractory unilateral limb pain syndrome, post-herpetic neuralgia and acute herpes zoster pain. Pain treatment can be carried out via electrical stimulation of the tibial nerve. An exemplary electrical signal can have an electrical current of 10 mA and a frequency of 100 Hz.
(ii) Rehabilitation—restoring function, for example, grasping for supporting tasks of daily living. Generation of electric signal towards nerves that directly or indirectly innervate various muscles. An exemplary electrical signal can have an electrical current of 15 mA and a frequency of 50 Hz.
(iii) Incontinence (fecal or urinary) and overactive bladder. An exemplary electrical signal can have an electrical current of 5 mA to 20 mA and a frequency of 20 Hz.
(iv) Treatment of epilepsy, depression, Alzheimer, anxiety, obesity, bulimia, tinnitus, obsessive compulsive disorder, hypertension, or heart failure, by, for example, stimulating the neck, face, chest or stomach adjacent to the vagus nerve or one of its branches. An exemplary electrical signal can have an electrical current of 10 mA and a frequency of 10 Hz.
(v) Treatment of cancer, tumors, such as prostate cancer, brain cancer, breast cancer. Treatment of cancer and tumors may be achieved in addition and/or together with chemotherapy treatment. The system and methods of this invention may enhance the chemotherapy process. An exemplary electrical signal can have an electrical current of 10 mA and a frequency of 10 Hz.
(vi) Regulation of the immune system for treating autoimmune diseases, reducing systemic inflammation. An exemplary electrical signal can have an electrical current of 10 mA and a frequency of 10 Hz.
(vii) Hair growth enhancement by stimulating the scalp. An exemplary electrical signal can have an electrical current of 5 mA and a frequency of 1 MHz.

One specific use for the system of the present invention is treatment of disorders that benefit from vagal nerve stimulation or one of its branches. Such disorders includes, but are not limited to, substance addiction, anxiety disorders, autism, bipolar disorders, cerebral palsy, chronic headaches, cognitive impairment associated with Alzheimer disease, coma, depression, eating disorders (e.g., anorexia and bulimia), essential tremor, fibromyalgia, heart failure, hemicrania continua, juvenile myoclonic epilepsy, migraine headaches, mood disorders, narcolepsy, obesity, obsessive-compulsive disorder, sleep disorder, tinnitus and Tourette's syndrome, hypomanic personality disorder or any other organic hypersomnia, tension type headache, alcohol-induced sleep disorders, drug-induced sleep disorders, episodic mood disorders, autistic disorder, obsessive-compulsive disorders, dysthymic disorder, alcohol dependence syndrome, drug dependence, nondependent abuse of drugs, anorexia nervosa, specific disorders of sleep of nonorganic origin, unspecified disorders of eating, tension headache, circadian rhythm sleep disorder, organic parasomnia, organic sleep disorders, essential and other specified forms of tremor, hemicrania continua, infantile cerebral palsy, migraine, cataplexy and narcolepsy, rheumatic heart failure, myalgia and myositis, sleep disturbances, polyphagia, mood, Parkinson's disease and headache, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear, disease (AIED), Autoimmune myocarditis, Autoimmune oophoritis, Autoimmune orchitis, pancreatitis, acute pancreatitis, Autoimmune pancreatitis, Autoimmune retinopathy, Autoimmune urticaria, Axonal & neuronal neuropathy (AMAN), Balo disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, Sepsis, bleeding disorders.

The treatment could be performed by other branches of the vagus or other nerves that have influence on the diseases. Either by being connected to the vagus or to the areas that control the disease.

Vagus nerve stimulation may activate the body's natural inflammatory reflex to dampen inflammation and improve clinical signs and symptoms. The inflammatory reflex is a neurophysiological mechanism that regulates the body's immune system. It senses infection, tissue injury and inflammation and relays this information to the central nervous system, which then reflexively increases neural signaling peripherally through the vagus nerve and splenic nerve that extensively innervate the spleen and other visceral organs. The signal is transmitted to T cells in the spleen, which in turn direct effector cells including monocytes and macrophages to reduce their production of the mediators that initiate and perpetuate inflammation.

Inflammation plays a significant role in acute and chronic diseases including rheumatoid arthritis, inflammatory bowel disease, psoriasis, diabetes, heart disease and multiple sclerosis.

This in one of its aspects, the present invention provides a system for electrical stimulation of a body tissue comprising:
(a) an array of microneedles, the microneedles formed from an electrically conductive material and adapted for puncturing a surface of the tissue;
(b) a power unit
(c) one or more surface electrodes configured to be connected to the power unit and deliver electrical stimulation to the body tissue;
(d) a processing unit configured to determine from a signal input to the processing unit any one or more of:
   if the microneedles in the microneedle array are in a predetermined position in the tissue; and
   if tissue surrounding one or more microneedles has been ablated.

One or more of the surface electrodes may be wettable electrodes.

The system according of the invention may further comprise one or more force detectors, each force detector being configured to detect application of a pressure exerted by the tissue on one or more of the microneedles in the array, the pressure being indicative of a position of the microneedles in the tissue, and to generate a signal indicative of a position of one or more of the microneedles, the signal being input to the processing unit. One or more of the force detectors may comprise a displacement mechanism that activates the force detector when a force is detected by the force detector. The force detector may be a spring based detector in which an extent of compression of the spring is indicative of the pressure exerted by microneedles in the array. The force detector may also be a load cell-based force detector. One or more of the force detectors may comprise a switch that is closed when a predetermined force is detected by the force detector, and closing of the switch generates an electric signal input to the processing unit.

The processing unit may be configured to monitor the time dependent electric signal and determining if the microneedles in the microneedle array are in a predetermined position in the tissue may involve determining when the pressure exerted by the tissue on the microneedles is above a predetermined threshold.

The system of the invention may further comprise a switching circuit configured for intermittent electrical connection of one or more selectable subsets of the microneedles to the power unit and for intermittent electrical connection of one or more of the surface electrodes to the power unit.

The system of the invention may further comprise a signal generator, and the processing unit may be further configured to activate the signal generator to deliver an electrical signal between a selectable first subset of microneedles and a selectable second subset of microneedles or between a selectable first set of microneedles and at least one of the surface electrodes. The processing unit may be further configured to activate the signal generator to deliver an ablation signal between a first selectable subset of microneedles and either a second selectable subset of microneedles and one or more of the surface electrodes, the ablation signal selected to cause ablation of the tissue surrounding one or more of the microneedles. The processing unit may be further configured to activate the signal generator to deliver the ablation signal only after the processing unit has determined that the microneedle array is in the predetermined position. The processing unit may be further configured to activate the signal generator to generate a test signal between a first selectable subset of microneedles and either a second selectable subset of microneedles and one or more of the surface electrodes, and to determine if tissue surrounding the microneedles in the first selectable subset has been ablated involves analyzing a response of the tissue to the test signal. The processing unit may be further configured to generate the test signal between a plurality of first selectable subsets of microneedles and either one or more second selectable subsets of microneedles and one or more of the surface electrodes, and to determine if ablation of the microneedles in the array meets a predetermined criterion. The processing unit may be further configured to determine a number of microneedles in the array for which tissue surrounding the microneedle has been ablated. The predetermined criterion may be that the number of microneedles in the array for which tissue surrounding the microneedle has been ablated is above a predetermined threshold. The processing unit may be configured to deliver the electrical stimulation to the tissue when the predetermined criterion is met.

The ablation signal may be a voltage signal. The ablation signal may consist of a series of wave train pulses. The ablation signal may comprise a square voltage pulses of alternating sign, or a sine wave signal. The ablation signal may have, for example, an amplitude of 200 volts to 800 volts and a frequency of 1 KHz to 1,000 KHz.

Determining if tissue surrounding the microneedles in the first selectable subset has been ablated may involve monitoring a current or impedance response of the tissue and determining if the current is above a predetermined threshold or the impedance is below a predetermined threshold.

The system of the invention may be configured to deliver electrical stimulation through skin. In this case, the predetermined position of the microneedles is when the microneedles have penetrated through the stratum corneum layer of the skin. The system may be configured to deliver electrical stimulation to one or more nerves under the skin surface, such as a vagus nerve.

A method for electrical stimulation of a body tissue comprising:
(a) applying an array of microneedles to a surface of the body tissue, and puncturing the tissue surface to generate micropores in the tissue;
(b) applying one or more surface electrodes to the tissue surface over the micropores and delivering electrical stimulation through the one or more surface electrodes;
(c) determining any one or more of:
   if the microneedles in the microneedle array are in a predetermined position in the tissue; and
   if tissue surrounding one or more microneedles has been ablated.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B show a system for electrical stimulation of a body tissue in accordance with one embodiment of the invention; schematically illustrate one embodiment of the present system showing the control unit, surface electrodes and the microneedle array (FIG. 1A) and the control unit and surface electrodes applied to tissue with one surface electrode mounted over the micropores formed by the microneedles array (FIG. 1B);

FIG. 2 shows microneedle array that may be used in the system of FIG. 1 pressed onto a body tissue;

FIGS. 3A-B show electrical current between various combinations of microneedles in the microneedle array of FIG. 2, and FIG. 3C shows the shape of an individual microneedle;

FIGS. 4A-B schematically illustrate a vagus nerve stimulation procedure using the present system;

FIGS. 5A-B illustrate various signal profiles for micropore verification, ablation and treatment using the present system;

FIGS. 6-9 show graphs illustrating the levels of various cytokines in rats treated with the present system and rats challenged with LPS and treated with the present system;

FIG. 10 shows a graph showing changes in rat blood pressure following application of a stimulatory signal using the present system; and

FIG. 11 shows a method of electrical stimulation of a body tissue in accordance with one embodiment of this aspect of the invention.

### DETAILED DESCRIPTION

The present invention provides a system and method for electrical stimulation of a body tissue, which can be used to deliver an electrical signal to tissue using one or more surface electrodes. The system of the present invention employs a microneedle array and an ablation signal for forming electrically conductive micropores in a tissue and a processing unit for verifying that the micropores formed exhibit desired electrical conductivity characteristics.

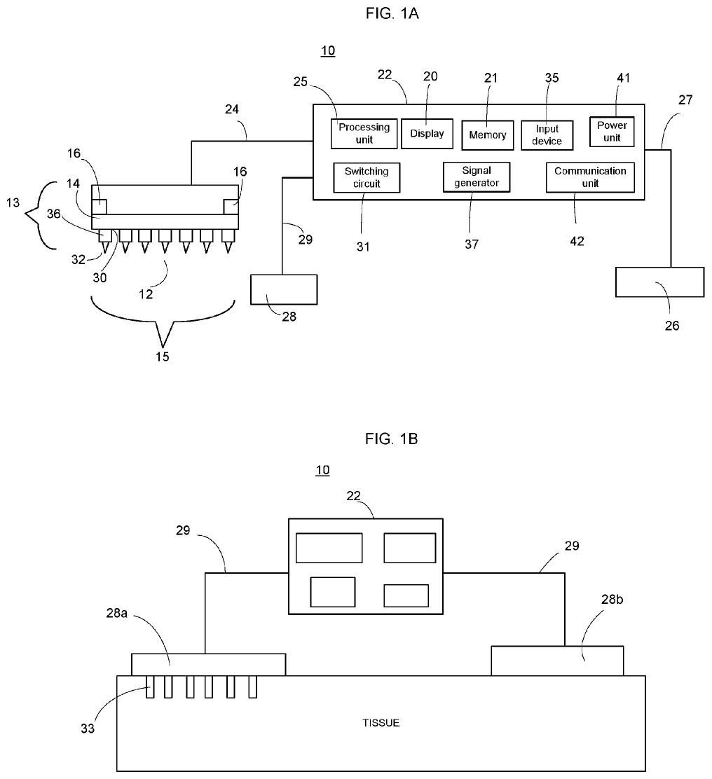

Referring now to the drawings, FIG. 1A illustrates a system 10 for electrical stimulation of a body tissue in accordance with one embodiment of the invention. The system 10 includes an applicator 13 including an array 15 of two or more microneedles 12 attached to a support 14.

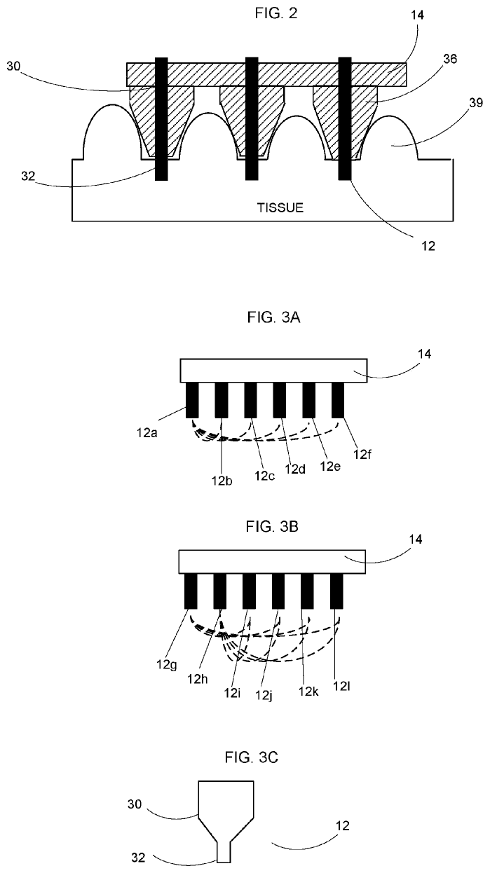

The support 14 may be fabricated, for example, from polycarbonate. The microneedles 12 are made from an electrically conductive material such as stainless steel, and, in addition to being able to puncture the tissue surface and enter the tissue, the microneedles 12 also function as electrodes, as explained in detail below. Each microneedle 12 may have a coating of an insulation material 36, such as polycarbonate, that prevents leakage of electric current through the sides of the microneedle, promotes conduction of an electrical current to the microneedle tip 32 and creates folds 39 (FIG. 2) to enhance penetration of microneedles.

In one embodiment, each microneedle 12 has a base portion 30 (surrounded by the insulation material 36 in FIG. 1A) and a pointed tip portion 32 shaped to puncture a first tissue and penetrate into the tissue to form micropores in a superficial layer of the first tissue. The shape of the insulation material 36 may be conic, in general, (FIG. 2) with a smaller diameter near the tip portion of the microneedle 32. The base portion 30, may be, cylindrical, extending about 10-10,000 micrometers in length and 10-500 micrometers in diameter. When utilized for puncturing skin over a region of the vagus nerve, for example, the length of the tip portion of the microneedle 32 can be 40-150 micrometers in length. The base portion 30 may also have a shape and diameter that are different than the tip 32. The diameter may be enlarged to allow a more stable base. For example, the microneedle may also have a non-cylindrical shape (e.g., a flat microneedle), with a width of about 40-300 micrometers and a length of about 40-8,000 micrometers and a height of extending about 10-10,000 micrometers (FIG. 3C). The microneedle array 12 may cover an area of about 0.5-5 square centimeters, typically 0.5-2 square centimeters for placement, for example, on the neck for vagus nerve stimulation.

The microneedle array 15 can be, for example, a 16×16 microneedle array. The microneedles can be bonded to the support 14 or held in place by friction, for example, by being inserted into sockets in the support 14. Alternatively, the support 14 can be fabricated integrally with the microneedles 12, for example, using semiconductor chip fabrication technologies, well known in the art.

The system 10 further includes a control unit 22, which includes a processing unit 25 and memory 21, a signal generator 37, power unit 41, and a switching circuit 31. The power unit 41 can include power components known in the art such as a transformer. The processing unit 25 controls the switching circuit 31, to intermittently connect the various electrodes of the system to poles of the signal generator 37 and/or to the power unit 41 and/or to the processing unit 25 during the various stages of the treatment, as explained in detail below. The switching circuit 31 may include more than one set of switches to connect the various electrodes. For example, one set could be used to connect the microneedles 12 to the processing unit 25 and/or the signal generator 37 and/or to the power unit 41 while another set is used to connect the surface electrodes 28 to the processing unit 25 and/or the signal generator 37 and/or to the power unit 41. The signal generator 37 may include more than one set of signal generators for the different signals. The power unit 41 may include more than one set power units for the different signals. The control unit 22 and/or the switching circuit 31 intermittently connects a selectable first subset of microneedles 12 in the array 15 to one pole of the power unit 41 and to intermittently connect a selectable second subset of microneedles 12 in the array 15 to a second pole of the power unit 41. The processing unit 25 can also control the signal generator 37, to generate an electrical signal between two poles of the power unit 41. The microneedles 12 can be connected to the switching circuit 31 via wires 24 or through a printed circuit (PCB). The components of the control unit 22 may be energized from a battery or from an external power source.

The control unit 22 may also include a display 20 and a user input device 35 such as a keypad or a touch screen which may be integral with the display 20. The input device 35 may be used to program the processing unit 25 to drive the switching circuit 31 and the signal generator 37 to generate an electric signal between selected first and second subsets of microneedles 12 and to generate an electric signal between selected first and second surface electrodes 28 having desired characteristics with regard, for example, to the frequency, electrical potential, and profile of the signal. The control unit 22 may also include a communication unit 42 to allow a remote control of the control unit 22, for example by Bluetooth.

The applicator 13 may include a force detector 16 that detects pressure applied to the tissue surface by the tips 32 of the microneedles 12 as the applicator 13 is pressed into the tissue surface during penetration of the microneedles 12 into the tissue. The microneedle array 15 may include a displacement mechanism that activates the force detector 16 when the microneedles 12 are pressed onto the tissue. The force detector 16 may generate a time dependent electric signal indicative of the pressure that is input to the processing unit 25. The force detector 16 can be a spring based transducer in which an extent of compression of the spring is indicative of the pressure applied to the tissue surface. Alternatively, the force transducer 16 may be a load cell-based transducer. The load-cell transducer may be connected to the display 20 of system 10 to provide an indication of a force applied by tip/s 32. A galvanic separation between the microneedles 12 and the control unit 22 may be maintained until a predefined force has been applied by the tip/s 32. The applicator 13 may include more than one set of force detectors 16. The force can be measured collectively or at each microneedle. A predefined force applied on the force detector may be a trigger for initiation of the ablation signal by microneedles 12, and also for verification that at least the predefined force is applied during the delivery of the ablation signal to the first tissue.

The system 10 further comprises a treatment surface electrode 28 and a ground surface electrode 26. The electrodes 26 and 28 are surface electrodes connected to the switching circuit 31 via wires 27 and 29, respectively. Wires 27 and 29 may include more than one wire each to connect to more than one surface electrode 26 and more than one surface electrode 28. The electrodes 26 and 28 can be dry, wet or wettable electrodes. A conductive liquid, such as saline can be used to wet the electrode surface. The saline may have a concentration, for example, between 0.1%-25%, preferably in the range of 5%-15%. The electrodes 26 and 28 can have any shape and dimensions as required in any application. For example, the electrodes 28 and 26 may be a 2×2 cm square with a width of 0.5 centimeters, or any other dimension and shape suitable for the desired treatment region, such as a circle or oval. The electrodes 26 and 28 can be disposable or reusable, and can include an adhesive surface for attachment to a first tissue (e.g., skin). The processing unit 25 controls the switching circuit 31 to intermittently connect two or more surface electrodes 28 to the poles of the power unit 41 and/or the signal generator 37. The processing unit 25 also controls the switching circuit 31 to connect one or more surface electrodes 26 to the poles of the power unit 41 and/or the signal generator 37.

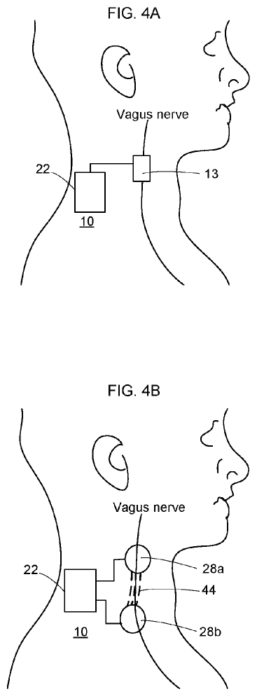

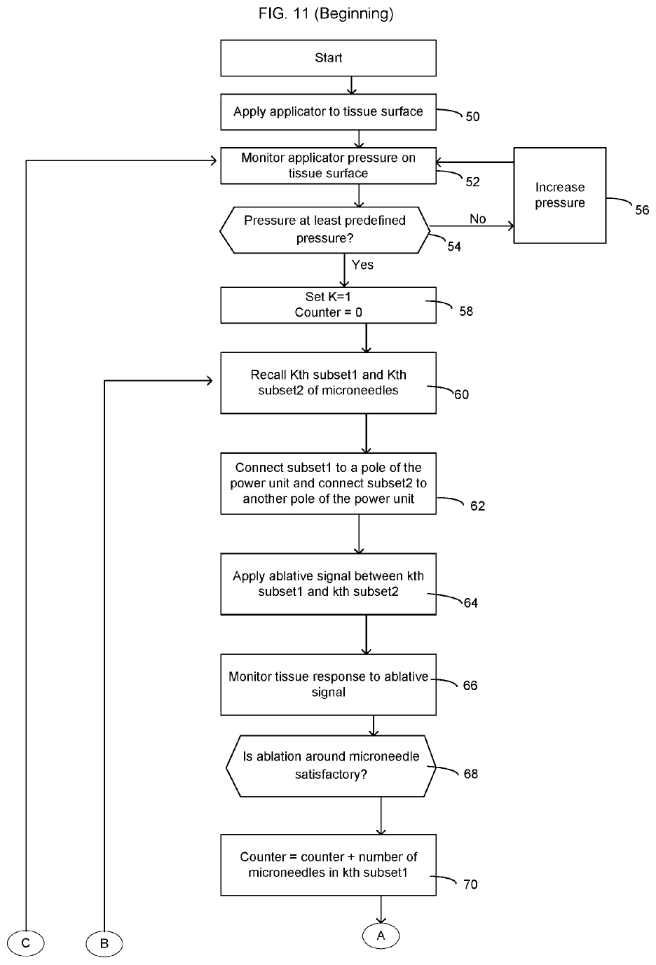

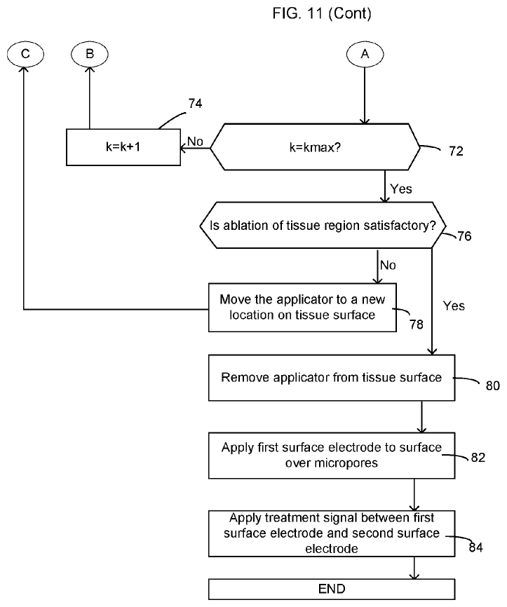

FIG. 11 shows a flow chart for a method of electrical stimulation of a tissue, using the system of the present invention, in accordance with one embodiment of the invention. In step 50, the applicator 13 is applied to a first tissue surface on top of a second tissue region to be treated. The second tissue may be a nerve, but not limited, that is located relatively deep (e.g., more than 0.5 centimeter, may also be less deep as well) under the surface of the first tissue (e.g., skin). FIG. 4A shows, as an example, the placement of the applicator 13 on the neck of an individual over the vagus nerve in a treatment for stimulating the vagus nerve (the vagus nerve may be located at a depth of more than 1 centimeter under the skin). As pressure is applied by the applicator 13, the force detector 16 detects a pressure applied to the tissue surface and generates an electrical signal indicative of the pressure that is input to the processing unit 25. The processing unit 25 constantly monitors or is triggered by the signal input from the force transducer 16 (step 52) and determines whether the force of the applicator 13 on the tissue surface is at least a threshold pressure that was previously stored in a memory of the processing unit (step 54). In an alternative embodiment, not shown, when a pressure on the tissue surface is detected by the force detector 16, the force detector 16 closes a switch which sends a signal to the processing unit 25 that a pressure has been detected. As shown in FIG. 2, pressure of the applicator 13 on the tissue surface can cause the tissue to create deformations 39 and be inserted into the spaces between the microneedles 12 and its coating 36. This deformation has been found to be helpful for microneedle 12 penetration into the superficial layer of the first tissue and may also reduce the pressure needed for insertion of the microneedles through the tissue surface.

If the pressure of the applicator 13 on the tissue surface has not yet reached the predefined pressure, the pressure of the applicator on the tissue surface is increased (step 56) and the process returns to step 52. When the pressure of the applicator 13 on the tissue surface is at least the predefined pressure, this is an indication that the microneedles 12 are applying the requisite pressure and the process can proceed to step 58. The inventors have found that a pressure in the range of 0.3 to 2 kg/cm², but typically in the range of 0.5 to 1.2 kg/cm², is indicative of the microneedles being in the requisite position. In the case of skin, the microneedles 12 are applying the requisite pressure when the microneedle tips 32 are pressed through the stratum corneum layer of the skin.

Additionally or alternatively some or all of the microneedles 12 may be connected to a pole of the signal generator 37 via the switching circuit 31, and the ground electrode 26 applied to the tissue surface and connected to another pole of the signal generator 37 via the switching circuit 31. A DC or pulsatile signal of non-stimulating energy (e.g., 1 V or below 2 mA) is generated by the signal generator 37 generating an electrical signal between the microneedles 12 and the ground electrode 26. The current is monitored by the processing unit 25 and the tissue impedance is calculated from the electrical signal. A tissue impedance that is below a predetermined threshold is indicative of the formation of micropores, and that the requisite pressure is being applied.

When it is determined that the requisite pressure is being applied, an index k is set to 1, and a counter is set to 0 (step 58). This is only an example and other methods known in the art to measure time and activate switching circuit may be used. Now, in step 60, a kth subset1 of microneedles in the microneedle array 15 and a kth subset2 of microneedles in the microneedle array 15 (previously stored in the memory 21) are recalled from the memory 21 of the control unit 22. The kth subset1 and the kth subset2 are non-empty disjoint sets of microneedles. The microneedles in the kth subset1 are then connected to a first pole of the signal generator 37 and/or power unit 41 via the switching circuit 31 and the microneedles in the kth subset2 are then connected to another pole of the signal generator 37 and/or power unit 41 via the switching circuit 31 (step 62).

In an alternate embodiment, a kth subset2 is not used, and instead, the ground electrode 26 is attached to the second pole of the signal generator.

The process now continues with step 64 where a kth ablation signal, generated by the signal generator 37 is applied between the microneedles in the kth subset1, on the one hand, and the microneedles in the kth subset2, on the other hand. The kth ablation signal is designed to cause ablation of the tissue surrounding the microneedles in subset1. FIG. 3A shows schematically, as a first example, part of the microneedle array 15 having 6 microneedles. In the example of FIG. 3A, microneedle 12a is the sole microneedle in the kth subset1. The remaining five microneedles, microneedles 12b to 12f constitute the kth subset2. The dashed curves in FIG. 3A indicate an electrical signal between microneedle 12a and each of the microneedles in the kth subset2, upon application of the ablation signal to the first tissue. FIG. 3B shows schematically, as a second example, part of the microneedle array 15 having 6 microneedles in which the microneedle 12g together with microneedle 12h constitute kth subset1. The remaining four microneedles, microneedles 12i to 12l constitute the kth subset2. The dashed curves in FIG. 3B indicate an electric signal between microneedles 12g and 12h and each of the microneedles in the kth subset2, upon application of the ablation signal. This is only an example and any permutation of subset1 and subset2 are allowed.

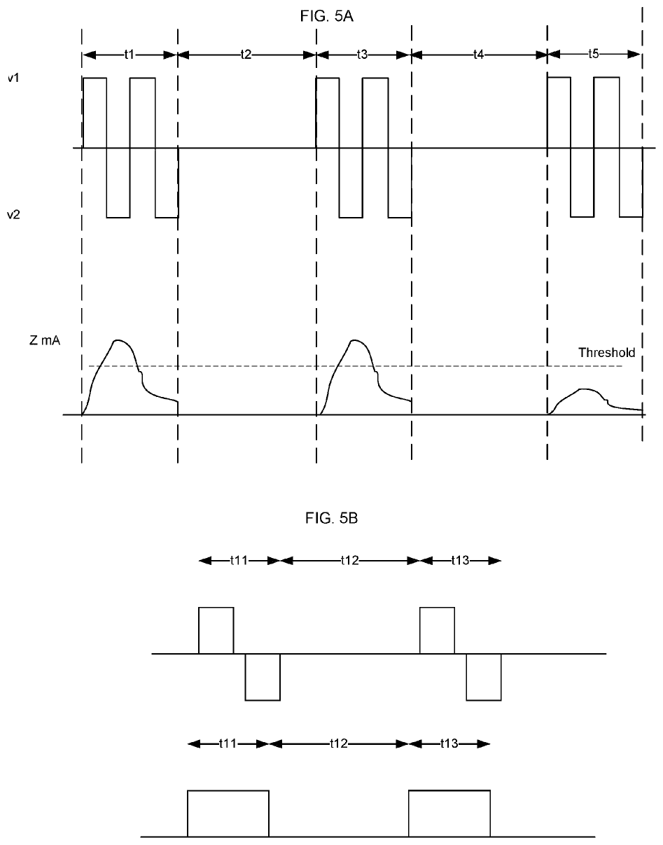

FIG. 5, upper panel, shows an example of an ablation signal applied between the microneedles in the kth subset1 and the microneedles in the kth subset2 to the first tissue. The ablation signal shown in the upper panel of FIG. 5 is a voltage signal consisting of a series of wave train pulses, where each pulse consists of a number of square voltage pulses of alternating sign. The square shape is only an example and other shapes could be used (e.g., a sine waveform can also be used). Depending on the tissue being treated, each pulse would typically have an amplitude of 25 V to 2,000 V (typically 200-800 v), and a frequency of 1 KHz to 1,000 KHz.

The pulse trains of the ablation signal are applied during time intervals of duration t1, t3, t5, etc. These times may be predefined or determined on the fly, but typically, the duration times of the pulses are between 1-20 milliseconds. The pulses are separated by periods of quiescence of duration t2, t4, etc. when the ablation signal is not applied and when impedance can be measured.

Referring again to FIG. 11, as the kth ablation signal is being applied between the kth subset1 and subset2, the pressing unit monitors the current flowing between the one or more of the microneedles in the kth subset1 and one or more of the microneedles in the kth subset2 (step 66). The bottom panel of FIG. 5 shows a typical current response to an ablation signal such as the ablative voltage signal shown in the upper panel. As the ablation progresses, the current rises indicating a decrease in the tissue impedance, penetration of the microneedles through the tissue, and the ablation process. The current reaches a peak and then decays. While not wishing to be bound by any particular theory, it is believed that at the current peak and then during the decay of the current, ablative tissue that is less conductive surrounds the microneedle. This may be an indication of the state of the micropore.

Then, in step 68, the processing unit 25 determines if the ablation around one or more of the microneedles in the kth subset1 is satisfactory. Satisfactory ablation of the tissue around the microneedles reduces the overall impedance of the tissue and reduces the amplitude of the treatment signal by surface electrodes 28 needed for effective treatment.

In one embodiment, the ablation around the microneedles in subset1 is determined to be satisfactory when the height of the peak in the current response is above a predetermined threshold level (indicated by the horizontal dashed line in the bottom panel, and previously stored in the memory 27). In another embodiment, a change (e.g., reduction) in the phase of the signal is used to determine if the ablation is satisfactory. If the ablation around the microneedles in the kth subset1 is satisfactory, the counter, which counts the number of microneedles whose surrounding tissue has been satisfactorily ablated, is increased by the number of microneedles in the kth subset1. (step 70).

The process now proceeds to step 72 where it is determined whether k is equal to a maximum value kₘₐₓ, where kₘₐₓ is the number of pairs of subset1 and subset2 which are to be examined for the ablation of surrounding tissue. If k is not equal to kₘₐₓ, then in step 74 k is increased by 1 and the process returns to step 60 where subset1 and subset2 for the new value of k is recalled from the memory 27. If k=kₘₐₓ, the process continues to step 76 where it is determined whether the overall ablation of the tissue is satisfactory. This is determined from the final value of the counter which counts the number of microneedles for which ablation of the surrounding tissue was found to be satisfactory. Overall ablation of the tissue may be considered satisfactory, for example, when the number of microneedles for which ablation of the surrounding tissue was found to be satisfactory is above a predetermined threshold. Otherwise, overall tissue ablation would not be considered to be satisfactory. The threshold may be, for example, a predetermined fraction, such as 0.8, of the number of microneedles in the array 15, (i.e. the overall ablation of the tissue is satisfactory when the ablation around at least 80% of the microneedles is satisfactory). When it is determined that ablation of the tissue region is not satisfactory, the applicator may be moved to a new location on the tissue surface (step 78) and the process can return to step 52 and begin again with the applicator at the new location. In another example not shown, another location for generation of micropores may be chosen for creation of micropores. The process of micropores creation could be performed in more than one location to allow placement of more than one surface electrode 28 over micropores (not included in FIG. 11).

If, at step 76, it is determined that ablation of the tissue region is satisfactory, the applicator 13 is removed from the tissue surface (step 80) and the treatment electrode 28 is placed on the tissue surface over the micropores that were formed in the tissue (step 82). The quality of the treatment requires good alignment of the surface electrode 28 with the formed micropores. This can be accomplished, for example, by marking the tissue surface with the boundaries of the area of the tissue surface to be treated, and applying the microneedle array 15 (step 50) and the treatment electrode 28 (step 82) within the boundary markings. Another method for assuring alignment of the treatment electrode 28 with the micropores makes use of a device, described in U.S. Pat. No. 10,688,301 having an aperture covered by a removable flap on which the treatment electrode 28 is incorporated. The device is adhered to the tissue surface with the aperture over the area of the tissue surface to be treated. The flap can be lifted or removed to reveal the skin surface under the aperture. The microneedle array 15 can be pressed into the tissue surface through the aperture of the patch, and the micropores thus formed can then be ablated and the microneedle array 15 removed and the flap, can then be closed to bring the surface electrode 28 into contact with the skin surface directly over the formed micropores. In another example the aperture does not include a flap, and the surface electrode is separate from the flap.

FIG. 1B shows the system 10 with the treatment electrode 28a and another electrode 28b placed on the tissue surface with the treatment electrode 28a over the micropores 33 that were formed in the first tissue. This is only an example and micropores could be created under surface electrode 28b as well. The micropores 33 can extend 10-300 micrometers (but not limited) into the tissue and in the case of skin preferably extend through the highly resistive keratinized layer. FIG. 4B shows, again as an example, the placement of the surface electrodes 28a and 28b on the neck of an individual in a treatment for stimulating the vagus nerve. A treatment procedure to modify the levels of cytokines is initiated by selecting the target vagus nerve (left and/or right along the neck of the subject) then marking the location for two surface electrodes 28 to be placed over the tissue surface over the vagus nerve with a suitable distance between electrodes (e.g., 1-10 cm). Other treatments such as hypertension could be performed as well, by other electrical signal parameters for treatment. The treatment electrode 28a is positioned over the micropores that were formed in the tissue over the vagus nerve (cf FIG. 4A). A treatment signal 44 is then generated between the surface electrode 28a passing through a second tissue such as the vagus nerve and the surface electrode 28b (step 84). The presence of the micropores under the electrode 28a tends to direct the current though deeper layers of the tissue, such as the vagus nerve in the arrangement shown in FIG. 4B. FIG. 5B illustrates the waveform of two exemplary electrical signals that may be used in the electrical stimulation of the tissue. These signals can be voltage or current controlled with a current of 0.1 mA-50 mA, a voltage of 0.1 V-100 V and a frequency of 1-1M Hz. The pulse duration can vary from 10-1,000 microseconds (typical 50-500 microseconds).

The process ends with the completion of the treatment.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non-limiting fashion.

### Example 1

### Rat Cytokine Study

The effect of electrical stimulation on pro- and anti-inflammatory cytokines was studied using a rat model and a prototype of the present system.

Thirty adult rats were divided to four groups:
(i) lipopolysaccharide (LPS);
(ii) lipopolysaccharide (LPS)+electrical stimulation treatment; and
(iii) electrical stimulation treatment only; and
(iv) control.

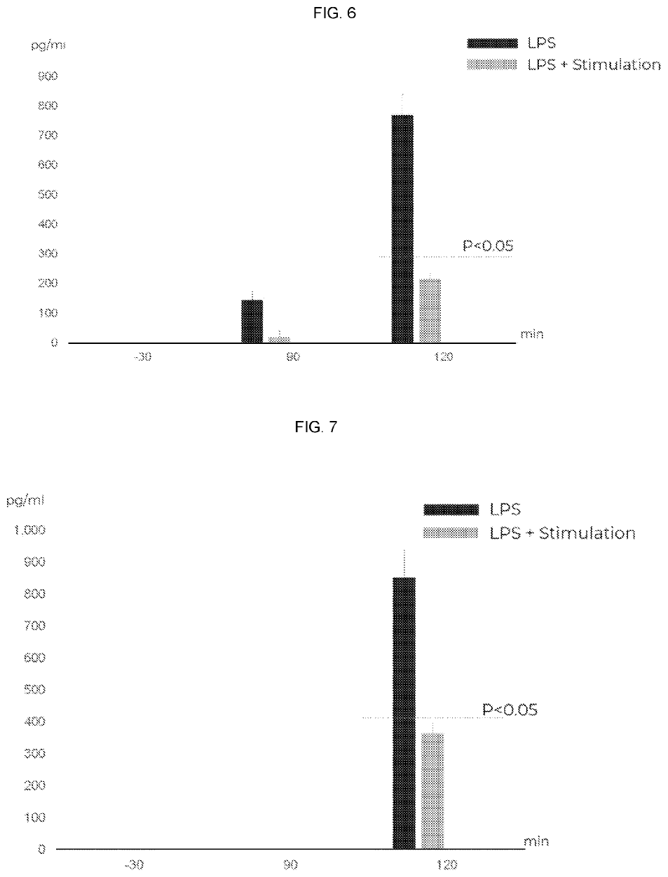

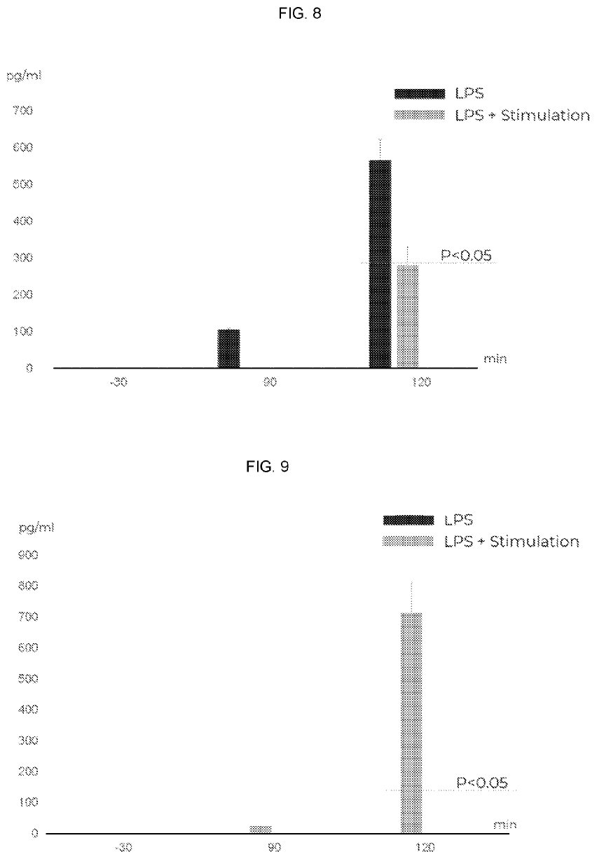

Lipopolysaccharide from Escherichia coli (LPS, 5 mg/kg body weight) was administrated via injection to the rat at time 0 minutes (FIGS. 6-9) and blood samples were collected 30 minutes before the LPS administration and 90 and 120 minutes following the LPS administration.

Electrical stimulation was performed using a tissue stimulating system of the present invention having a microneedle array with 80-160 microneedles and a surface area of 0.5-1.5 square centimeters) for generation of micropores and measurement of tissue characteristics and two wettable surface electrodes 0.5-2.0 centimeters in diameter for the delivery of the electrical stimulation and impedance measurement, and

Prior to treatment, the rat was shaved in an area of the neck with a hair trimmer while taking care not to break the skin surface. The treatment location was determined anatomically on the neck of the rat, over the vagus nerve and the insertion location of the microneedle array was marked on the skin. Tissue characteristics prior to and following micropore generation were monitored as described hereinabove.

Micropores were generated by pressing the microneedle array into the skin in the marked area of the skin surface over the vagus nerve. After insertion of the microneedle array into the skin, it was determined as described above, that the microneedles are in the requisite position with the microneedle tips having passed though the stratum corneum. The tissue surrounding each microneedle was then ablated as determined above, and the microneedle array removed from the skin surface. A treatment surface electrode was applied to the skin surface over the micropores and a ground electrode was place 1-10 centimeters away from the treatment electrode.

The rats in groups ii and iii were treated with a stimulatory signal having the following parameters:
Frequency—5-30 Hz
Pulse width—100-500 microseconds
Pulse shape—Bi polar
Interphase—10-100 microseconds
Intensity Range—1.0-4.0 mA

Groups ii and iii were treated with 2 sessions of 10 minutes each.

Groups iii was not administered with LPS.

Groups iv was not administrated with LPS and was not electrically treated.

The results are shown in FIGS. 6-9. FIGS. 6-8 describe the average measurement of the cytokines TNFα, IL6, IL-1β, respectively, and FIG. 9 describes an average measurement of IL-10 at 90 minutes and 120 minutes following administration of LPS. The bars indicate standard deviation.

(IL-1β: 145 pg/ml, and 770 pg/ml; IL6: below sensitivity level, 852 pg/ml; TNFα: 105 pg/ml, 565 pg/ml; IL-10: below sensitivity level, below sensitivity level) are of Group i and bars 3 and 4 (IL-1β: 20 pg/ml, 214 pg/ml; IL6: below sensitivity level, 364 pg/ml; TNFα: below sensitivity level, 280 pg/ml; IL-10: 53 pg/ml, 713 pg/ml) are of Group ii. Groups iii and iv were below detection sensitivity. The statistical calculations showed a reduction of the level of the TNFα, IL6, IL-1B cytokines in Group ii which was significant with p<0.05, and an increase of the level of the IL-10 in Group ii which was also significant with p<0.05.

### Example 2

### Rat Blood Pressure Study

The effect of electrical stimulation on rat blood pressure was studied on four rats using a system of the invention. After the formation of micropores in the skin of the neck, as described above, a stimulatory surface electrode was placed over the micropores and the vagus nerve was stimulated with a signal having the following parameters:
Frequency—10-40 Hz
Pulse width—200-1000 microseconds
Pulse shape—Bi polar
Interphase—10-100 microseconds
Intensity Range—0.5-4.0 mA

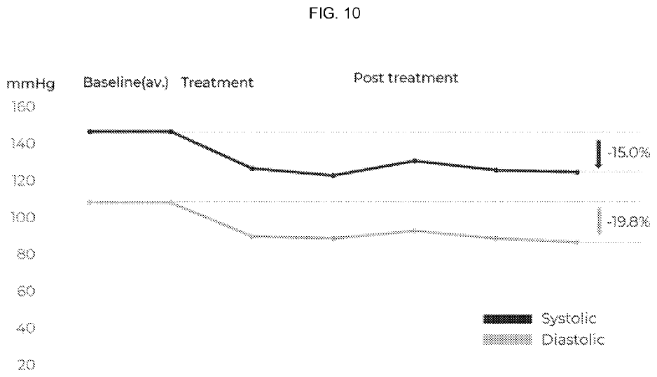

FIG. 10 shows the average systolic and diastolic blood pressure of the four rats showing an average baseline obtained over a 50 minute period prior to treatment. This was followed by the above treatment for 5 minutes, and then by 45 minutes of blood pressure monitoring. The systolic pressure decreased by 15% during the measurement period following treatment and the diastolic pressure decreased by about 20% during the same time period.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-29. (canceled)

30. A system for electrical stimulation of a body tissue comprising:
(a) an array of microneedles, said microneedles formed from an electrically conductive material and configured for puncturing a surface of the tissue;
(b) a power unit;
(c) one or more surface electrodes configured for delivering electrical stimulation to the body tissue from said power unit;
(h) a processing unit configured for determining if said microneedles are in a predetermined position in a tissue; and/or if said tissue surrounding one or more of said microneedles has been ablated.

31. The system according to claim 30, further comprising one or more force detectors configured to detect application of a pressure exerted by the tissue on one or more of said microneedles in said array and to generate an electrical signal indicative of said pressure.

32. The system according to claim 31, wherein said processing unit is configured to monitor said electrical signal over time so as to determine when said pressure exerted by said tissue on said microneedles is above a predetermined threshold.

33. The system according to claim 30, further comprising a switching circuit configured for intermittently connecting one or more subsets of said microneedles from said array to said power unit and for intermittently connecting one or more of said surface electrodes to said power unit.

34. The system according to claim 33, further comprising a signal generator activatable by said processing unit to deliver an electrical signal between a selectable first subset of microneedles and a selectable second subset of microneedles or between a selectable first set of microneedles and at least one of the surface electrodes.

35. The system according to claim 34, wherein said processing unit is further configured to activate said signal generator to deliver an ablation signal to at least said subset of said microneedles of said array.

36. The system according to claim 35, wherein said processing unit is further configured to activate the signal generator to deliver said ablation signal only after said processing unit has determined that said microneedle array is in the predetermined position.

37. The system according to claim 35, wherein said processing unit determines if tissue surrounding the microneedles has been ablated by analyzing a response of said tissue to a test signal.

38. The system according to claim 30, wherein said processing unit is further configured for determining a number of said microneedles surrounded by ablated tissue.

39. The system according to claim 35, wherein said ablation signal is a voltage signal.

40. The system according to claim 39, wherein said ablation signal has an amplitude of 200 to 800 volts.

41. The system according to claim 39, wherein the ablation signal has a frequency of 1 KHz to 1,000 KHz.

42. The system according to claim 30, wherein said predetermined position of said microneedles is through a stratum corneum layer of a skin.

43. The system according to claim 30, wherein said one or more surface electrodes is a wettable electrode.

44. A method for electrical stimulation of a body tissue comprising:
(a) applying an array of microneedles to a surface of the body tissue, and puncturing the tissue surface to generate micropores in the tissue;
(b) determining if said microneedles in said microneedle array are in a predetermined position in the tissue; and
(c) ablating tissue surrounding at least a subset of said microneedles of said array;
(d) removing said array of microneedles from said tissue; and
(e) applying one or more surface electrodes to a surface of said tissue over micropores formed by said microneedles and delivering electrical stimulation via said one or more surface electrodes.

45. The method according to claim 44, further comprising determining if said tissue surrounding one or more of said microneedles of said array has been ablated prior to (d).

46. The method according to claim 44, wherein ablating said tissue is carried out with a voltage signal having an amplitude of 200 to 800 volts and a frequency of 1 KHz to 1,000 KHz.
